(19) United States
(12) Patent Application Publication
(10) Pub. No.: US 2002/0127203 A1
(43) Pub. Date: Sep. 12, 2002

(54) Ribavirin-pegylated interferon alfa HCV combination therapy

(76) Inventor: Janice K. Albrecht, Winter Park, FL (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(21) Appl. No.: 09/981,215

(22) Filed: Oct. 16, 2001

Related U.S. Application Data
(60) Provisional application No. 60/241,557, filed on Oct. 18, 2000.

(51) Int. Cl. A61K 38/21

(52) U.S. Cl. 424/85.7; 514/43

(57) ABSTRACT

A method for treating patients having chronic hepatitis C infection to eradicate detectable HCV-RNA involving a combination therapy using a therapeutically effective amount of ribavirin and a therapeutically effective amount of pegylated interferon-alfa, e.g, pegylated interferon-alfa-2b or -2a for a time, i.e., at least about twenty-four weeks, sufficient to eradicate detectable HCV-RNA by the end of the treatment time period and to maintain no detectable HCV-RNA for at least 12 weeks after the end of the treatment time period is disclosed.

### BACKGROUND OF THE INVENTION

The present invention relates to methods of treating patients having chronic hepatitis C infection by administering a therapeutically weight-effective amount of ribavirin and a therapeutically effective amount of pegylated interferon-alfa for a treatment time period sufficient to eradicate detectable HCV-RNA and to maintain no detectable HCV-RNA for a period of at least twelve weeks after the end of the treatment time period.

Chronic infection with hepatitis C virus is an insidious and slow-progressing disease having a significant impact on the quality of life. It can eventually result in cirrhosis of the liver, decompensated liver disease and/or hepatocellular carcinoma.

International Publication No. WO98/48840 discloses the use of pegylated interferon-alfa to treat hepatitis C infections.

Nieforth, et al., (Clin. Pharmacol. Ther., 1996, 59:636-646) has reported a comparison of the in vivo activity of Roferon®A and a polyethylene glycol-modified Roferon®A in healthy volunteers. The results, however, suggested that the conjugates could not be administered less than twice weekly and therefore offered little therapeutic advantage over the unmodified counterpart.

Co-pending, commonly assigned U.S. patent application Ser. No. 08/742,305 discloses methods of administering polymer-cytokine conjugates to individuals susceptible to treatment with the cytokine. See also WO/00/37110. Neither reference discloses the method of this invention.

Polyethylene glycol modification of other proteins has been reported by Fuertges, et al., (Journal of Controlled Release, 1990, Vol.11:139-48).

Combination therapy of interferon alfa-2b and ribavirin to treat chronic hepatitis C for 24 weeks is disclosed by Reichard, et al., (Lancet 1998; 351; 83-87)

T. Poynard, et al., (Lancet, 1998, Vol. 352,1426-1432) disclose that treating chronic hepatitis C patients who had not been treated with interferon or ribavirin with 3 MIU of interferon alfa-2b TIW plus 1000-1200 mg of ribavirin per day for 48 weeks resulted in a sustained virological response at 24 weeks after treatment in 43% of the patients. See also J. G. McHutchinson, et aL, (N. Engl. J. Med., 1998, 339:1485-1492). G. L. Davis, et al., (N. Engl. J. Med. 339:1493-1499) disclose that treating chronic hepatitis C patients who relapsed after treatment with interferon with 3 million International Units (“MIU”) of interferon alfa-2b three times a week (“TIW”) plus 100-1200 mg of ribavirin per day for 48 weeks results in higher rates of sustained virologic response than treatment with interferon alone.

There is a need to provide an improved therapy for treating chronic hepatitis C patients to produce a sustained virological response at least twelve weeks after the end of treatment in a greater number of patients.

### SUMMARY OF THE INVENTION

The present invention provides a method of treating a patient having chronic hepatitis C infection that comprises administering to the patient a therapeutically weight-effective amount of ribavirin in association with a therapeutically effective amount of pegylated interferon alfa protein once a week for a treatment time period sufficient to eradicate detectable HCV-RNA and to maintain no detectable HCV-RNA for at least twelve weeks after the end of the treatment time period.

The present invention also provides a method of treating a patient having chronic hepatitis C infection that comprises administering to the patient a therapeutically weight-effective amount of ribavirin per day that is 800 mg/day for people having a weight of less than about 65 kg, 1000 mg/day for people having a weight in the range of about 65 kg to about 85 kg, and 1200 mg/day for people having a weight of about 85 kg or higher, and about 1.5 micrograms per kilogram of pegylated interferon alfa-2b protein the patient's body weight once a week for a treatment time period sufficient to eradicate detectable HCV-RNA and to maintain no detectable HCV-RNA for at least twelve weeks after the end of the treatment time period.

The present invention also provides a method of treating a patient having chronic hepatitis C infection that comprises administering to the patient at least about 10.6 mg/kg of the patient's body weight per day of ribavirin and in association with about 1.5 micrograms of pegylated interferon alfa-2b protein per kilogram of the patient's body weight once a week for a treatment time period sufficient to eradicate detectable HCV-RNA and to maintain no detectable HCV-RNA for at least twelve weeks after the end of the treatment time period.

### DETAILED DESCRIPTION

The present invention provides a method of treating patients having chronic hepatitis C infections that comprises administering a therapeutically weight-effective amount of ribavirin and a therapeutically effective amount of pegylated interferon-alfa protein for a treatment time period that is long enough to eradicate detectable HCV-RNA at least by the end of the treatment time period and to maintain no detectable HCV-RNA for at least twelve weeks after the end of the treatment time period. In a preferred embodiment of the present invention, the therapeutically effective amounts of both the ribavirin and the pegylated interferon alfa are dosed according to the weight of the patient. Thus, in a prefrred embodiment of the present invention, by administering about 1.5 micrograms of pegylated interferon alfa-2b protein per kilograms of the patient's body weight once a week (“QW”) and at least about 13 mg/kg of ribavirin per day the HCV-RNA is eradicated (i.e., lowered to less than 100 copies of HCV-RNA/ml of serum) during the treatment time period such that no detectable HCV-RNA level is detected at the end of the period and twelve weeks after the end of the treatment time period. The treatment time period is at least about 24 weeks, preferably at about 40-50 weeks, most preferably about 48 weeks.

Ribavirin, 1β-D ribofuranosyl-1H-1,2,4 triazole 3-carboxamide, also known as Rebetol®, available from ICN Pharmaceuticals, Inc., Costa Mesa, Calif., is described in the Merck Index, compound No. 8199, Eleventh Edition. Its manufacture and formulation is described in U.S. Pat. No. 4,211,771. The effective amount of ribavirin administered in the treatment time period is from about 800 mg to about 1600 mg per day, preferably about 800 mg to about 1400 mg/day, and most preferably about 800 mg/day, about 1000 mg/day or about 1200 mg/day depending upon the weight of the patient.

The term “therapeutically weigh-effective amount of ribavirin” means an amount that is sufficient to produce a sustained virologic response for at least about twelve weeks post treatment, preferably for at least about twenty-four weeks post treatment, most preferably forty eight weeks post treatment.

In a preferred embodiment of the present invention, therapeutically weight-effective amount of ribavirin is at least about 10.6 mg of ribavirin per kilogram of patient's body weight (“10.6 mg/kg of ribavirin per day”), preferably is in the range of at least about 13 mg/kg to about 14.5 mg/kg of ribavirin per day, preferably at least about 13 mg/kg of ribavirin per day. In another preferred embodiment, the preferred therapeutically weight-effective amount of ribavirin is about 800 mg/day for people having a weight of less than about 65 kg, about 1000 mg/day for people having a weight in the range of about 65 kg to about 85 kg, and about 1200 mg/day for people having a weight greater than about 85 kg.

The following preferred embodiments for administering pegylated interferon alfa are presented.

The term “pegylated interferon alfa” as used herein means polyethylene glycol modified conjugates of interferon alfa, preferably interferon alfa-2a and -2b. The preferred polyethyleneglycol-interferon alfa-2b conjugate is PEG₁₂₀₀₀-interferon alfa-2b. The phrases “12,000 molecular weight polyethylene glycol conjugated interferon alpha” and “PEG₁₂₀₀₀-IFN alfa” as used herein mean conjugates such as are prepared according to the methods of International Application No. WO 95/13090 and containing urethane linkages between the interferon alfa-2a or -2b amino groups and polyethylene glycol having an average molecular weight of 12000.

The preferred PEG₁₂₀₀₀-interferon alfa-2b is prepared by attaching a PEG polymer to the epsilon amino group of a lysine residue in the IFN alfa-2b molecule. A single PEG₁₂₀₀₀ molecule is conjugated to free amino groups on an IFN alfa-2b molecule via a urethane linkage. This conjugate is characterized by the molecular weight of PEG₁₂₀₀₀ attached. The PEG12000-IFN alfa-2b conjugate is formulated as a lyophilized powder for injection. The objective of conjugation of IFN alfa with PEG is to improve the delivery of the protein by significantly prolonging its plasma half-life, and thereby provide protracted activity of IFN alfa.

The terms “pegylated interferon alfa protein” and “micrograms of pegylated interferon alfa protein/kg” as used herein in reference to pegylated interferon alfa-2b means micrograms (μg) of interferon alfa-2b in the polyethyleneglycol modified conjugate of interferon alfa-2b per kilogram (“kg”) of patient's body weight.

The term “interferon-alfa” as used herein means the family of highly homologous species-specific proteins that inhibit viral replication and cellular proliferation and modulate immune response. Typical suitable interferon-alfas include, but are not limited to, recombinant interferon alfa-2b such as Intron-A® interferon available from Schering Corporation, Kenilworth, N.J., recombinant interferon alfa-2a such as Roferon® interferon available from Hoffmann-La Roche, Nutley, N.J., recombinant interferon alpha-2C such as Berofor alpha 2 interferon available from Boehringer Ingelheim Pharmaceutical, Inc., Ridgefield, Conn., interferon alpha-n1, a purified blend of natural alfa interferons such as Sumiferon® available from Sumitomo, Japan or as Wellferon® interferon alpha-n1 (INS) available from the Glaxo-Wellcome Ltd., London, Great Britain, or a consensus alpha interferon such as those described in U.S. Pat. Nos. 4,897,471 and 4,695,623 (especially Examples 7, 8 or 9 thereof) and the specific product available from Amgen, Inc., Newbury Park, Calif., or interferon alfa-n3 a mixture of natural alfa interferons made by Interferon Sciences and available from the Purdue Frederick Co., Norwalk, Conn., under the Alferon Tradename. The use of interferon alfa-2a or alpha 2b is preferred. Since interferon alpha 2b, among all interferons, has the broadest approval throughout the world for treating chronic hepatitis C infection, it is most preferred. The manufacture of interferon alpha 2b is described in U.S. Pat. No. 4,530,901.

The effective amount of pegylated interferon alfa protein that is administered in the treatment time period is In the range of about 0.5 to about 9 micrograms of pegylated interferon alfa-2b protein per kilogram of body weight (“μg/kg”) once a week (QW), and preferably is in the range of about 1.5 μg/kg to about 9 μg/kg QW for at least about twenty-four to about forty-eight weeks, most preferably about 1.5 μg/kg of pegylated interferon alfa-2b, QW for about forty-eight weeks.

When the pegylated interferon-alfa administered is a pegylated interferon alfa-2a, the therapeutically effective amount of pegylated interferon alfa-2a administered during the treatment in accordance with the present invention, including in first and second treatment time periods, is in the range of about 50 micrograms to about 500 micrograms per week, preferably about 150 micrograms to about 250 micrograms per week, or preferably about 180 micrograms to about 250 micrograms per week or preferably about 150 micrograms to about 180 micrograms per week or most preferably about 180 micrograms per week, or alternatively the effective amount is in the range of about 50 micrograms to about 500 micrograms once a week(“QW”), preferably about 150 micrograms to about 250 micrograms QW, or preferably about 180 micrograms to about 250 micrograms QW or preferably about 150 micrograms to about 180 micrograms QW or most preferably about 180 micrograms QW or alternatively the effective amount is in the range of about 25 micrograms to about 250 micrograms twice a week (“BIW”), preferably about 75 micrograms to about 125 micrograms BIW, preferably about 75 micrograms to about 125 micrograms BIW, or preferably about 75 micrograms to about 90 micrograms BIW, or most preferably about 90 micrograms BIW.

Other interferon alfa conjugates can be prepared by coupling an interferon alfa to a water-soluble polymer. A non-limiting list of such polymers include other polyalkylene oxide homopolymers such as polypropylene glycols, polyoxyethylenated polyols, copolymers thereof and block copolymers thereof. As an alternative to polyalkylene oxide-based polymers, effectively non-antigenic materials such as dextran, polyvinylpyrrolidones, polyacrylamides, polyvinyl alcohols, carbohydrate-based polymers and the like can be used. Such interferon alfa-polymer conjugates are described in U.S. Pat. Nos. 4,766,106, 4,917,888, and 5,792,834, European Patent Application No. 0 236 987, European Patent Application Nos. 0510 356, 0593 868 and 08098 996, pegylated interferon-alfa -2a and International Publication Nos. WO 95/13090 and WO/64016.

Pharmaceutical compositions of pegylated interferon-alfa suitable for parenteral administration may be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g. human serum albumin), toxicity agents (e.g. NaCl), preservatives (e.g. thimerosol, cresol or benzylalcohol), and surfactants (e.g. tween or polysorbates) in sterile water for injection. The pegylated interferon alfa may be stored as lyophilized powders under a refrigeration at 2° C.-8° C. The reconstituted aqueous solutions are stable when stored between 2° C. and 8° C. and used within 24 hours of reconstitution. See for example U.S. Pat. Nos. 4,492,537, 5,762,923 and 5,766,582. PEG-Intron(peginterferon alfa 2b) is available from Schering Corporation, Kenilworth, N.J., and PEGASYS(Peginterferon alfa-2a) is available from Hoffmann La Roche, Nutley, N.J.

The term “patients having chronic hepatitis C infections” as used herein means any patient having chronic hepatitis C and includes treatment naive patients, relapsers and non-responders.

These patients having chronic hepatitis C include those who are infected with multiple HCV genotypes including type 1 as well as those infected with, inter alia, HCV genotypes 2 and/or 3 as well as HCV genotypes 2, 3, 4, 5 and/or 6 and other possible HCV genotypes.

The term “treatment naive patients” as used herein means patients with chronic hepatitis C who have never been treated with ribavirin or any interferon, including but not limited to interferon-alfa, or pegylated interferon-alfa.

The term “relapsers” as used herein means patients with chronic hepatitis C who have relapsed after initial response to previous treatment with interferon alone or in combination with ribavirin.

The term “non-responders” as used herein means patients with chronic hepatitis C who have not responded to prior treatment with any interferon alone or in combination with ribavirin.

A person suffering from chronic hepatitis C infection may exhibit one or more of the following signs or symptoms:

(a) elevated ALT,

(b) positive test for anti-HCV antibodies,

(c) presence of HCV as demonstrated by a positive test for the presence of HCV-RNA in the serum,

(d) clinical stigmata of chronic liver disease,

(e) hepatocelluar damage.

To practice the invention, the combination therapy of pegylated interferon-alfa and ribavirin is administered to the patient exhibiting one of more of the above signs or symptoms in the treatment time period in amounts sufficient to eliminate or at least alleviate one or more of the signs or symptoms.

Ribavirin is administered to the patient in association with pegylated interferon-alfa, that is, the pegylated interferon-alfa dose is administered during the same period of time that the patient receives doses of ribavirin. Pegylated interferon-alfa formulations are not effective when administered orally, so the preferred method of administering the pegylated interferon-alfa is parenterally, preferably by subcutaneous, IV or IM injection. Ribavirin may be administered orally in capsule or tablet form in association with the parenteral administration of pegylated interferon-alfa. Of course, other types of administration of both medicaments, as they become available are contemplated, such as by nasal spray, transdermally, by suppository, by sustained release dosage form, and by pulmonary inhalation. Any form of-administration will work so long as the proper dosages are delivered without destroying the active ingredient.

The term “no detectable HCV-RNA” in the context of the present invention means that there are fewer than 100 copies of HCV-RNA per ml of serum of the patient as measured by quantitative, multi-cycle reverse transcriptase PCR methodology. HCV-RNA is preferably measured in the present invention by the methodology described below. This methodology is referred to herein as HCV-RNA/qPCR. The lower limit of detection of HCV-RNA is 100 copies/mL.

RNA is extracted from patient serum using a guaninidium thiocyanate-phenol-chloroform mister followed by ethanol-ammonium acetate precipitation. The precipitated RNA is centrifuged and the resulting pellet is dried in a Centrivap console (Labconco, Kansas City, Mo.). The dry pellet is then re-suspended in 30 microliters of an Rnasin (Promega Corp., Madison, Wis.), dithiothritol, and diethylpyrocarbonate-treated water mixture. Samples are kept at or below −20° C. (preferably below −70° C.) until RNA reverse transcription (RT) and PCR.

In order to convert the entire RNA sequence into cDNA in the RT reaction, random hexadeoxyribonucleotides (Pharmacia Biotech, Piscataway, N.J.) are used as primers for the first strand cDNA synthesis. Two aliquots of 3 microliters of re-suspended sample are added to 3 microliters of 100 ng/μl random primers and denatured at 70° C., then reverse transcribed at 40° C. for one hour using M-MLV reverse transcriptase (USB, Cleveland, Ohio) in standard buffer containing 5 mM MgCl₂. The final RT reaction volume is 26 μl. The PCR is started immediately following the reverse transcription.

A modified version of the PCR method is performed using heat-stable Taq polymerase to amplify the cDNA. Seventy-five microliters of PCR mix is added to the entire RT reaction volume (26 μl) to a final MgCl₂ concentration of 1.5 mM in a total volume of 101 μl. Each 101 μl sample is then split into 50.5 μl and a layer of mineral oil is placed on top to prevent evaporation.

The PCR cycle consists of annealing for 90 sec., extension for 90 sec., and denaturation for 90 sec., at 55° C., 74° C. and 94° C., respectively. Thermocycling samples are submitted to a final 74° C. extension for 10 minutes. Four different cycle sets are used. By loading the sample in duplicate, and splitting these samples evenly after RT, there are four tubes from one sample. Each of the four tubes is given a different cycle number, enhancing sensitivity and accuracy in the quantitation process. The thermocycling efficiency will be assessed by satisfactory amplification of known copy number RNA standards included in each set of 60 tubes. Two primer sets are used for the amplification, both from the 5′ untranslated region of the HCV genome. Both of these primer sets are highly conserved and detect all known subtypes of HCV. Primer set 1: upstream 5′-GTG GTC TGC GGA ACC GGT GAG T-3′, downstream 5′-TGC ACG GTC TAC GAG ACC TC-3′ which produces a 190 bp product. Primer set 2: upstream 5′-CTG TGA GGA ACT ACT GTC TTC-3′, downstream 5′-CCC TAT CAG GCA GTA CCA CAA-3′ which produces a 256 bp product.

The amplified cDNA is then electrophorised in 3% agarose gel and transferred to nylon membrane. The target DNA is detected by Southern blotting and immunostaining using a nonradioactive digoxigenin-labeled DNA probe. These procedures are performed using automated instruments for PCR thermocycling, agarose gel electrophoresis, vacuum-transfer Southern blot, hybridization, and immunostaining. Each membrane contains known copy number serially diluted standards that are used to construct standard curves for quantitative measurement of the specimen bands. Originally standard curves are made from carefully diluted HCV-RNA from transcribed clones. Radioactive incorporation studies, gel electrophoresis, and OD 260 are performed on the transcripts to determine that they are of the expected length. After the production of the RNA transcripts quantitated clone standards called “pooled” standards are generated which better represent the heterogeneous nature of HCV, one would encounter in natural infection. These pools are made by combining large amounts of serum or plasma from known infected individuals. The serum/plasma pools are calibrated with PCR, against the clone transcripts and then diluted in the known PCR-negative fluids. Finally, the higher copy number samples of the pools are checked against the cDNA Quantiplex nucleic acid detection system from Chiron Inc. (Emeryville, Calif.). These “double quantitated” pools are aliquoted and saved at −70° C. Dilutions of 5,000,000, 1,000,000, 500,000, 100,000, 10,000, and 1000 copies/ml are used in each experiment.

Each Southern blot membrane is scanned into a computer using an automated scanner/densitometer, at intervals during development to determine when the standard curve is most linear. The resultant electronic images are then measured for band area and mean band density. All of the reading are standardized to integrated band density and compared to the standard curve to obtain a numerical value of viral copy number for each band.

The term “sustained virologic response” as used in the context of the present invention means that there is no detectable HCV-RNA in the patients treated in accordance with the present invention for at least twelve weeks after the end of the combined therapy treatment. Preferably, the period of sustained virologic response will be at least twenty four weeks, and more preferably at least one year—or longer—after the end of treatment. For HCV genotyping, INNO-L PA HCV (Innogenetics, Zeijmaurde, Belgium) second generation assay may be used.

The following clinical protocol may be used to administer the combination therapy of the present invention.

Study Design

Chronic Hepatitis C: Peg-Intron(pegylatedinterferon-alfa-2b) Plus REBETOL(ribavirin) vs. REBETRON

1529 patients were randomized equally to three treatment regimens for a treatment time period of 48 weeks. The three treatment regimens are:

1) Peg-Intron 1.5 μg/kg/QW plus 800 mg/day ribavirin

2) Peg-Intron 1.5 μg/kg to 0.5 μg/kg/QW plus 1000-1200 mg/day ribavirin

3) REBETRON: Intron A(interferon alfa-2b) 3 MIU TIW plus 1000-1200 mg/day ribavirin.

Thus, in regimen 1, patients received 1.5 μg/kg of Peg-Intron once weekly in association with 800 mg/day of ribavirin for 48 weeks. In regimen 2, patients received 1.5 μg/kg Peg-Intron once weekly in combination with 1000 to 1200 mg/day of ribavirin for four weeks followed by Peg-Intron 0.5 μg/kg once weekly in combination with 1000 to 1200 mg/day of ribavirin for forty-four weeks. Finally, in regimen 3 patients received 3 million International Units of Intron A three times a week in combination with 1000 to 1200 mg/day of ribavirin.

The primary efficacy endpoint for the study is the sustained loss of serum HCV-RNA twelve weeks post treatment and the results presented below were obtained at twelve weeks post treatment. Prior studies have demonstrated that the results of the study at twelve weeks post treatment are similar to the results at twenty-four weeks post treatment within 1 to 2%.

The following tables summarize the data analyzed by treatment and by weight adjusted dose of ribavirin within the treatment group.

Response For All Treated Patients and by HCV 1 vs HCV 2/3

Table 1 summarizes the overall results of the three treatment regimens. s15 As can be seen in Table 1, Peg-Intron 1.5 μg/kg plus 800 mg/day of Ribavirin obtained a successful response in 54% of the patient population, whereas therapy regimens 2 and 3 both obtained a significantly lower response rate of 47%. Thus, Peg-Intron 1.5 mg/kg/800 mg Ribavirin is significantly more effective than both Peg-Intron 1.5 to 0.5 μg/kg/1000-1200 mg Ribavirin and Rebetron (p=0.01).

HCV genotype is the most significant predictor of response to therapy. Approximately 70% of patients in the U.S. and Europe are genotype 1. As for all treated patients, Pegintron 1.5 μg/kg/800 mg Ribavirin is more effective for treating HCV 1. It should be noted that patients with genotype 2 or 3 generally responded better to all forms of therapy than patients with genotype 1.

TABLE 1
Peg-Intron plus Rebetol vs Rebetron Sustained loss of
HCV 12 Weeks Following the End of Treatment

| | Peg-Intron | | |
|---|---|---|---|
| | 1.5 μg/kg + | Peg-Intron | Rebetron: |
| | 800 mg | 1.5 → 0.5 μg/kg + | Intron A 3MIU TIW + |
| HCV | Rebetol | 1000-1200 mg | 1000-1200 mg Rebetol |
| Genotype | (Ribavirin) | Rebetol (Ribavirin) | (Ribavirin) |
| All | 54%* | 47% | 47% |
| Genotypes | | | |
| HCV 1 | 41% | 34% | 33% |
| HCV 2/3 | 84% | 79% | 79% |

*All genotypes p = 0.0125 Peg 1.5 μg/kg/QW + 800 mg/day ribavirin vs. Rebetron
*All genotypes p = 0.016 Peg 1.5 μg/kg/QW + 800 mg/day ribavirin vs. Peg 1.5 μg/kg?QW − >0.5 μg/kg/QW + 1000-1200 mg/day ribavirin

Effect of HCV Genotype and Baseline HCV Level

Baseline HCV level can also have a significant effect on a patient's response within a genotype. Patients with genotype 1 that had a high virus load have the lowest response rate. High virus load is defined as having greater than 2 million copies of HCV RNA/ml of serum. In the Rebetron registration studies, the difference in response rate between patients with low virus load and high virus load was 6%. Low virus load is defined as having less than or equal to 2 million copies of HCV RNA/ml of serum.

TABLE 2
Peg-Intron plus Rebetol vs Rebetron Effect of
HCV Genotype and Baseline HCV Level Sustained Loss of
HCV 12 Weeks Following the End of Treatment

| | | Peg-Intron | Rebetron: |
|---|---|---|---|
| | | 1.5 → | Intron A |
| HCV Genotype | Peg-Intron | 0.5 μg/kg + | 3MIU TIW + |
| and Pretreatment | 1.5 μg/kg + | 1000-1200 mg | 1000-1200 mg |
| HCV RNA Level | 800 mg Rebetol | Rebetol | Rebetol |
| (copies/ml) | (Ribavirin) | (Ribavirin) | (Ribavirin) |
| HCV 1 | 41% | 34% | 33% |
| HCV 2/3 | 84% | 79% | 79% |

As can be seen, Peg-Intron 1.5 μg/kg QW plus 800 mg/day Ribavirin demonstrated superior results in both the low virus load and high virus load populations vis-a-vis treatment regimens 2 and 3.

Effect of Patient Body Weight on Response

At the 24 week in treatment analysis it was observed that patient body weight appears to have an effect on loss of HCV-RNA response, particularly in the PegIntron 1.5 μg/kg/QW plus 800 mg/day Ribavirin group. The range of body weights for the patients in the study was large (38-181 kg) with the majority of patients (63%) weighing greater than 75 kg. The results of the study were re-analyzed (Tables 3, 4 and 5) and the response rate was determined based on the mg/kg of ribavirin that the patient received (patient body weight/ribavirin dose). As shown in Table 3, the response rate is related to both the dose of Peg-Intron on a μg/kg/QW basis and the dose of ribavirin on a mg/kg/day basis.

TABLE 3
Peg-Intron plus Rebetol vs Rebetron Sustained Loss of HCV
12 Weeks Following the End of Treatment
All Genotypes Response by mg/kg of Ribavirin

| | | Peg-Intron | REBETRON: |
|---|---|---|---|
| | Peg-Intron | 1.5 → 0.5 μg/ | Intron A |
| | 1.5 μg/kg + | kg + | 3MIU TIW + |
| | 800 mg | 1000-1200 mg | 1000-1200 mg |
| | Rebetol | Rebetol | Rebetol |
| Ribavirin mg/kg | (Ribavirin) | (Ribavirin) | (Ribavirin) |
| All Ribavirin | 54% | 47% | 47% |
| doses | | | |
| ≦10.6 mg/kg | 50% (161/323) | 40% (14/35) | 29% 7/24 |
| >10.6-13.2 | 59% (76/129) | 43% (57/132) | 41% (51/123) |
| mg/kg | | | |
| >13.2 mg/kg | 66% (39/59) | 49% (171/350) | 50% (177/357) |

The 10.6 mg/kg dose of ribavirin is about 800 mg/day(795 mg/day)in a 75 kg person in the Peg-Intron 1.5 μg/kg/QW plus 800 mg/day group; only 37% of patients in treatment regimen received this dose and the remainder received less. In contrast, the majority of the other two treatment groups received more than 10.6 mg/kg ribavirin. Thus, by increasing the dose of ribavirin/kg of the patient's body weight there is demonstrated an unexpectedly better increase in efficacy of 66% that is most pronounced in the Peg-Intron 1.5 μg/kg/QW plus 800 mg Ribavirin therapy compared to the efficacies of 49% and 50% in treatment groups 2 and 3.

Table 4 shows the respective response by HCV gentotype. As is evident, patients having HCV genotype 1 receive the most benefit from increasing the dose of Peg-Intron and the dose of ribavirin. Efficacy of the Peg-Intron 1.5 μg/kg/QW plus 800 mg Rebetol (ribavirin) regimen increased substantially as the Peg-Intron μg/kg dose and the ribavirin mg/kg doses were increased both within the patient population receiving this treatment and relative to the other therapy regimens.

TABLE 4
Peg-Intron plus Rebetol vs Rebetron Sustained Loss of
HCV 12 Weeks Following the End of Treatment
HCV vs HCV2/3 Response by mg/kg of Ribavirin

| | | Peg-Intron | Rebetron |
|---|---|---|---|
| | Peg-Intron | 1.5 → 0.5 μg/ | Intron A |
| | 1.5 μg/kg + | kg + | 3MIU TIW + |
| | 800 mg | 1000-1200 mg | 1000-1200 mg |
| Ribavirin mg/kg | Rebetol | Rebetol | Rebetol |
| HCV 1 | | | |
| All Ribavirin | 41% | 34% | 33% |
| Doses | | | |
| ≦10.6 mg/kg | 38% (85/226) | 26% (6/23) | 24% (4/17) |
| >10.6-13.2 | 46% (39/84) | 32% (31/96) | 22% (18/81) |
| mg/kg | | | |
| >13.2 mg/kg | 53% (20/38) | 35% (80/229) | 38% (92/245) |
| HCV 2/3 | | | |
| All Body Weights | 84% | 79% | 79% |
| <10.6 mg/kg | 82% (73/89) | 73% (8/11) | 50% (3/6) |
| >10.6-13.2 | 87% (33/38) | 74% (25/34) | 79% (33/42) |
| mg/kg | | | |
| >13.2 mg/kg | 90% (18/20) | 81% (88/109) | 81% (79/97) |

Table 5 summarizes the response by HCV genotype and baseline HCV-RNA virus load. For patients with HCV genotype 1 and high virus load treatment with Peg-Intron 1.5 μg/kg and ribavirin >13.2 mg/kg, there is an improved response in this difficult to treat population.

TABLE 5
Sustained Loss of HCV 12 Weeks Following the End of
48 Week Treatment. Effect of HCV Genotype

| | | Peg-Intron | Rebetron: |
|---|---|---|---|
| | Peg-Intron | 1.5 → 0.5 μg/ | Intron A |
| | 1.5 μg/kg + | kg + | 3MIU TIW + |
| | 800 mg | 1000-1200 mg | 1000-1200 mg |
| | Rebetol | Rebetol | Rebetol |
| Ribavirin mg/kg | (Ribavirin) | (Ribavirin) | (Ribavirin) |
| HCV 1 ≦2 | | | |
| Million | | | |
| All Ribavirin | 71% | 51% | 45% |
| Doses | | | |
| ≦10.6 mg/kg | 70% (38/54) | 20% (1/5) | 33% (1/3) |
| >10.6-13.2 | 61% (17/28) | 56% (15/27) | 27% (3/11) |
| mg/kg | | | |
| >13.2 mg/kg | 100% (10/10) | 51% (36/70) | 48% (39/82) |
| HCV 2/3 | | | |
| All Body Weights | 31% | 26% | 29% |
| ≦10.6 mg/kg | 27% (47/172) | 28% (5/18) | 21% (3/14) |
| >10.6-13.2 | 39% (22/56) | 23% (16/69) | 21% (15/70) |
| mg/kg | | | |
| >13.2 mg/kg | 36% (10/28) | 28% (44/159) | 33% (53/163) |
| HCV 2/3 <2 | | | |
| Million | | | |
| All | 91% | 78% | 77% |
| ≦10.6 mg/kg | 89% (24/27) | 50% (1/2) | 50% (1/2) |
| >10.6-13.2 | 89% (16/18) | 58% (7/12/) | 69% (9/13) |
| mg/kg | | | |
| >13.2 mg/kg | 100% (8/8) | 85% (39/46) | 82% (31/38) |
| HCV 2/3 >2 | | | |
| Million | | | |
| All | 80% | 79% | 80% |
| ≦10.6 mg/kg | 79% (49/62) | 78% (7/9) | 50% (2/4) |
| >10.6-13.2 | 85% (17/20) | 82% (18/22) | 83% (24/29) |
| mg/kg | | | |
| >13.2 mg/kg | 71% (5/7) | 78% (49/63) | 81% (48/59) |

This enhancement of efficacy included all aspects of the disease will result in:

Sustained eradication of detectable HCV-RNA;

Improvement in hepatic inflammation;

Normalization of ALT;

Improvement in HQL.

Many modifications and variations of this invention can be made without departing from its spirit and scope, as will be apparent to one skilled in the art. The specific embodiments described herein are offered by way of example only, and the invention is to be limited only by the terms of the appended claims along with the full scope of equivalents to which such claims are entitled.

1. A method of treating a patient having chronic hepatitis C infection that comprises administering to the patient a therapeutically weight-effective amount of ribavirin in association with a therapeutically effective amount of pegylated interferon alfa protein for a treatment time period sufficient to eradicate detectable HCV-RNA and to maintain no detectable HCV-RNA for at least twelve weeks after the end of the treatment time period.

2. The method of claim 1, wherein the therapeutically weight-effective amount of ribavirin administered is from about 800 mg to about 1400 mg per day.

3. The method of claim 2, wherein the therapeutically weight-effective amount of ribavirin administered is about 800 mg/day, about 1000 mg/day or about 1200 mg per day.

4. The method of claim 1, wherein the patient that has chronic hepatitis C is infected with multiple HCV genotypes.

5. The method of claim 1, wherein the patient that has chronic hepatitis C is infected with HCV genotype 1.

6. The method of claim 1, wherein the patient that has chronic hepatitis C is infected with HCV genotype 2 and/or 3.

7. The method of claim 1, wherein the pegylated interferon alfa that is administered is selected from the group consisting of interferon alfa-2a, interferon alfa-2b, interferon alfa-2c, interferon alfa n-1, interferon alfa n-3 and consensus interferon.

8. The method of claim 1, wherein the pegylated interferon alfa that is administered is pegylated interferon alfa-2a or pegylated interferon alfa-2b.

9. The method of claim 1, wherein the pegylated interferon alfa that is administered is a pegylated interferon alfa-2b and wherein the amount of pegylated interferon alfa-2b that is administered in the treatment time period is about 1.5 micrograms per kilogram of pegylated interferon alfa-2b protein per week on a weekly basis for at least twenty-four weeks.

10. The method of claim 9, wherein the pegylated interferon alfa-2b is administered on a weekly basis for about forty-eight weeks.

11. The method of claim 1, wherein the therapeutically weight-effective amount of ribavirin to be administered is in an amount that is at least about 10.6 mg/kg of the patient's body weight per day.

12. The method of claim 1, wherein the patient that has chronic hepatitis C is infected with HCV genotype 1, and wherein the patient has less than two million copies of hepatitis C virus per milliliter of the patient's serum.

13. The method of claim 12, wherein the therapeutically weight-effective amount of ribavirin to be administered per day is at least about 10.6 mg/kg of the patient's body weight per day.

14. The method of claim 1, wherein the patient that has chronic hepatitis C is infected with HCV genotype 1, and wherein the patient has greater than two million copies of hepatitis C virus per milliliter of the patient's serum.

15. The method of claim 14, wherein the therapeutically weight-effective amount of ribavirin to be administered per day is at least about 10.6 mg/kg of the patient's body weight per day.

16. The method of claim 1, wherein the patient that has chronic hepatitis C is infected with HCV genotype 2 and/or 3, and wherein the patient has less than two million copies of hepatitis C virus per milliliter of the patient's serum.

17. The method of claim 16, wherein the therapeutically weight-effective amount of ribavirin to be administered per day is at least about 10.6 mg/kg of the patient's body weight per day.

18. The method of claim 1, wherein the patient that has chronic hepatitis C is infected with HCV genotype 2 and/or 3, and wherein the patient has greater than two million copies of hepatitis C virus per milliliter of the patient's serum.

19. The method of claim 18, wherein the therapeutically weight-effective amount of ribavirin to be administered per day is at least about 10.6 mg/kg of the patient's body weight.

20. A method of treating a patient having chronic hepatitis C infection that comprises administering to the patient a therapeutically weight-effective amount of ribavirin of about 800 mg/day for a patient having a weight of about 60 to about 65 kg, about 1000 mg/day for a patient having a weight in the range of greater than about 65 kg to less than about 85 kg, and about 1200 mg/day for a patient having a weight greater than about 85 kg, in association with about 1.5 micrograms per kilogram of pegylated interferon alfa-2b protein once a week for a treatment time period sufficient to eradicate detectable HCV-RNA and to maintain no detectable HCV-RNA for at least twelve weeks after the end of the treatment time period.

21. The method of claim 20, wherein the patient that has chronic hepatitis C is infected with multiple HCV genotypes.

22. The method of claim 20, wherein the patient that has chronic hepatitis C is infected with HCV genotype 1.

23. The method of claim 20, wherein the patient that has chronic hepatitis C is infected with HCV genotype 2 and/or 3.

24. The method of claim 20, wherein the treatment time period is about forty weeks to fifty weeks.

25. The method of claim 20, wherein the patient that has chronic hepatitis C is infected with HCV genotype 1, and wherein the patient has less than two million copies of hepatitis C virus per milliliter of the patient's serum.

26. The method of claim 20, wherein the patient that has chronic hepatitis C is infected with HCV genotype 1, and wherein the patient has greater than two million copies of hepatitis C virus per milliliter of the patient's serum.

27. The method of claim 20, wherein the patient that has chronic hepatitis C is infected with HCV genotype 2 and/or 3, and wherein the patient has less than two million copies of hepatitis C virus per milliliter of the patient's serum.

28. The method of claim 20, wherein the patient that has chronic hepatitis C is infected with HCV genotype 2 and/or 3, and wherein the patient has greater than two million copies of hepatitis C virus per milliliter of the patient's serum.

29. A method of treating a patient having chronic hepatitis C infection that comprises administering to the patient at least about 10.6 mg/kg of the patient's body weight of ribavirin per day in association with about 1.5 micrograms/kg of the patient's body weight of pegylated interferon alfa-2b protein once a week for a treatment time period sufficient to eradicate detectable HCV-RNA and to maintain no detectable HCV-RNA for at least twelve weeks after the end of the treatment time period.

30. The method of claim 29, wherein the patient that has chronic hepatitis C is infected with multiple HCV genotypes.

31. The method of claim 29, wherein the patient that has chronic hepatitis C is infected with HCV genotype 1.

32. The method of claim 29, wherein the patient that has chronic hepatitis C is infected with HCV genotype 2 and/or 3.

33. The method of claim 29, wherein the treatment time period is at least about twenty-four weeks long.

34. The method of claim 29, wherein the treatment time period is about forty-eight weeks long.

35. The method of claim 29, wherein the patient that has chronic hepatitis C is infected with HCV genotype 1, and wherein the patient has less than two million copies of hepatitis C virus per milliliter of the patient's serum.

36. The method of claim 35 wherein the treratment time period is about forty-eight weeks.

37. The method of claim 29, wherein the patient that has chronic hepatitis C is infected with HCV genotype 1, and wherein the patient has greater than two million copies of hepatitis C virus per milliliter of the patient's serum.

38. The method of claim 37, wherein the treatment time period is about forty-eight weeks.

39. The method of claim 29, wherein the patient that has chronic hepatitis C is infected with HCV genotype 2 and/or 3, and wherein the patient has less than two million copies of hepatitis C virus per milliliter of the patient's serum.

40. The method of claim 39, wherein the treatment time period is about forty-eight weeks.

41. The method of claim 29, wherein the patient that has chronic hepatitis C is infected with HCV genotype 2 and/or 3, and wherein the patient has greater than two million copies of hepatitis C virus per milliliter of the patient's serum.

42. The method of claim 41, wherein the treatment time period is about forty-eight weeks.
